(19) United States
(12) Patent Application Publication
(10) Pub. No.: US 2012/0213332 A1
(43) Pub. Date: Aug. 23, 2012

(54) RADIATION THERAPY SYSTEM WITH A TELESCOPIC ARM

(76) Inventors: Franz Dirauf, Ebensfeld (DE); Franz Fadler, Hetzles (DE)

(21) Appl. No.: 13/397,507

(22) Filed: Feb. 15, 2012

(30) Foreign Application Priority Data
Feb. 16, 2011 (DE) DE102011004224.5

(51) Int. Cl. A61B 6/02; A61N 5/10

(52) U.S. Cl. 378/62

(57) ABSTRACT

A radiation therapy system includes a radiation therapy module having a cantilever arm, from which a therapeutic treatment beam is directable onto an object. The cantilever arm is secured on a kinematic control such that the cantilever arm may be rotated about an isocenter such that the therapeutic treatment beam may be directed from different angles onto the isocenter. The radiation therapy system includes an X-ray imaging device with at least one X-ray source and an X-ray detector. The X-ray source is configured as a single-tank X-ray generator and arranged in the cantilever arm such that X-radiation may be directed in a direction of the therapeutic treatment beam. A first end of a first telescopic arm is arranged rotatably on the cantilever arm. The X-ray detector is arranged on a second end of the first telescopic arm such that a diagnostic X-ray image of the object may be produced.

This application claims the benefit of DE 10 2011 004 224.5, filed on Feb. 16, 2011.

### BACKGROUND

The present embodiments relate to a radiation therapy system with a therapeutic radiation therapy module and at least one diagnostic X-ray imaging module.

The use of radiation to destroy diseased tissue is a widely used method in therapeutic medicine. Systems that employ high-energy, electromagnetic radiation (e.g., X-radiation, gamma radiation) or particle radiation (e.g., electrons, protons, carbon ions) are used. The radiation used for radiation therapy may be in the megavolt (MV) energy range. During radiation therapy, precise positioning of the patient is provided such that the body region to be irradiated (e.g., a tumor to be irradiated) is exposed to a sufficiently high radiation dose, but healthy tissue of the patient is damaged as little as possible. For the purposes of positioning, localization of the body region to be irradiated in the body of the patient may be performed during the treatment at regular time intervals. This may be performed using imaging X-ray procedures with radiation in the kilovolt (kV) energy range (e.g., using computed tomography). To avoid incorrect positioning of the patient, an examination of this kind may be performed directly in the irradiation position.

During irradiation treatments with irradiation from different directions, it is important that the treatment beam for each of the directions hits the tumor. The beams should intersect at a point lying in the region of the tissue to be irradiated. This point is the isocenter and represents the intersection of the beams corresponding to different irradiation positions.

DE 10 2010 034 101 A1 discloses a radiation therapy system based on the principle of in-line imaging with a kV-imaging apparatus permitting movement of the radiation unit in five degrees of freedom. In-line imaging (e.g., in-beam imaging) provides that the radiation unit may be used to generate radiation that may be used for diagnostic purposes. The generated radiation may be detected by a detector after penetration of the object. In this way, the therapeutic and diagnostic beams are substantially parallel and are supplied from the same source. In addition, the source may be adapted for low-energy radiation (e.g., kV-region) by, for example, using a carbon target instead of a tungsten target. This procedure is also described in the document “In-Line kView Imaging” from Siemens AG.

FIG. 1 shows a radiation therapy system with X-ray imaging modules according to the prior art that permits in-line imaging. A radiator head 3 is arranged on a cantilever arm 2, and both a therapeutic MV beam and a diagnostic kV-beam may be generated via this head. A holding arm 4 embodied as a C-arm is attached to the cantilever arm 2. The holding arm 4 includes a first articulated joint 5, to which an articulated arm section 6 is connected. The articulated arm section 6 is connected to a second articulated joint 7, on which an X-ray detector 8 for detecting the kV beams emitted by the radiator head 3 is arranged. The second articulated joint 7 permits the tilting of the X-ray detector 8. The cantilever arm 2 is arranged on a kinematic apparatus 9, which permits movement of the radiator head 3 in five degrees of freedom.

According to a further development of in-line imaging, an anti-parallel beam is used instead of a parallel diagnostic beam. The X-ray detector is replaced by an X-ray tube. The X-ray detector used for the detection of the diagnostic X-rays is arranged in a region of an outlet of the therapeutic beams. This imaging may be inverse-in-line imaging or inverse-in-beam imaging. After the performance of the diagnostic irradiation, the X-ray detector is removed from the beam path by, for example, swinging the X-ray detector out of the way.

The drawback of this embodiment of a radiation therapy system is that a patient lying on a patient table is freely accessible to the medical assistants from only one side of the patient table, while accessibility from the other side of the patient table is greatly restricted by the holding arm with the X-ray imaging module.

### SUMMARY AND DESCRIPTION

The present embodiments may obviate one or more of the drawbacks or limitations in the related art. For example, an improved radiation therapy system is provided.

In one embodiment, a radiation therapy system includes a radiation therapy module with a cantilever arm, from which a therapeutic treatment beam may be directed onto an object to be irradiated. The cantilever arm is secured on a kinematic control such that the cantilever arm may be rotated about an isocenter, thus enabling the therapeutic treatment beam to be directed from different angles onto the isocenter. The radiation therapy system also includes an X-ray imaging device with at least one X-ray source and an X-ray detector. The X-ray source is arranged in the cantilever arm such that X-radiation may be directed in a direction of the therapeutic treatment beam. A first end of a first telescopic arm is arranged rotatably on the cantilever arm. At a second end of the first telescopic arm, the X-ray detector is arranged such that a diagnostic X-ray image of the object (e.g., a patient) may be produced. The advantage of this is that, after taking one or more X-ray images, due to the variability of the telescopic arm length, the first telescopic arm with the X-ray detector moves away from the imaging position and is, for example, put into a park position. The patient being examined is freely accessible to the medical assistant personnel.

In another embodiment, a radiation therapy system includes a radiation therapy module with a cantilever arm, from which a therapeutic treatment beam may be directed onto an object to be irradiated. The cantilever arm is secured on a kinematic control such that the cantilever arm may be rotated about an isocenter, thus enabling the therapeutic treatment beam to be directed from different angles onto the isocenter. The radiation therapy system also includes an X-ray imaging device with an X-ray source and an X-ray detector. The X-ray detector is arranged movably on the cantilever arm and may be swung into an outlet area of the therapeutic treatment beam. A first end of a first telescopic arm is arranged rotatably on the cantilever arm. At a second end of the first telescopic arm, the X-ray source is arranged such that a diagnostic X-ray image of the object (e.g., a patient) may be produced when the X-ray detector is swung into the outlet area of the treatment beam. The first telescopic arm may advantageously also be used with known radiation therapy systems with X-ray apparatuses with inverse-in-line imaging or inverse-in-beam imaging.

In yet another embodiment, a radiation therapy system includes a radiation therapy module with a cantilever arm, from which a therapeutic treatment may be directed onto an object to be irradiated. The cantilever arm is secured on a kinematic control such that the cantilever arm may be rotated about an isocenter, thus enabling the therapeutic treatment beam to be directed from different angles onto the isocenter. The radiation therapy system also includes an X-ray imaging device with an X-ray source and an X-ray detector. A first telescopic arm and a second telescopic arm are arranged on a base carrier with a rotatable connecting element. The X-ray detector is arranged at an end of the first telescopic arm that faces away from the connecting element. The X-ray source is arranged at an end of the second telescopic arm that faces away from the connecting element. The X-ray detector and the X-ray source may be positioned such that a diagnostic X-ray image of the object may be produced. The advantage of this that the telescopic arms used for positioning the X-ray imaging components are not connected to the individual technical feature of the cantilever arm of the radiation therapy system.

In one embodiment, the first telescopic arm and the second telescopic arm may each include at least two arm members connected to each other by articulated joints. This allows one or more arm members to be swung.

In another embodiment, lengths of the first telescopic arm and the second telescopic arm may be variable.

In addition, the first telescopic arm may be rotatable about a first axis standing perpendicular to a first securing point of the first telescopic arm on the cantilever arm. The X-ray imaging component arranged on the first telescopic arm may be positioned such that a diagnostic X-ray image of the object may be produced.

In one embodiment, the connecting element may be rotatable about a second axis standing perpendicular to a second securing point of the connecting element on the base carrier. The X-ray imaging components arranged on the first telescopic arm and the second telescopic arm may be positioned such that a diagnostic X-ray image of the object may be produced.

In an advantageous embodiment, the base carrier may be arranged on the base and firmly connected to the base.

### BRIEF DESCRIPTION OF THE DRAWINGS

FIG. 1 shows a radiation therapy system with X-ray imaging modules according to the prior art;

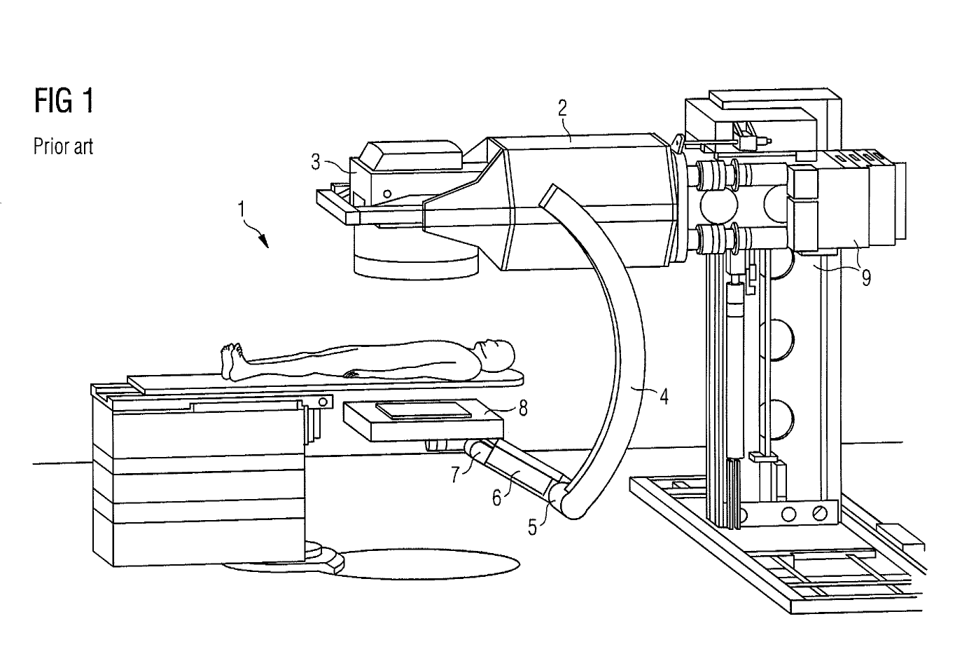

FIG. 2 shows one embodiment of a radiation therapy system with a first telescopic arm having an X-ray detector;

FIG. 3 shows one embodiment of a radiation therapy system with a first telescopic arm having an X-ray tube; and

FIG. 4 shows one embodiment of a radiation therapy system with a first telescopic arm and a second telescopic arm.

### DETAILED DESCRIPTION OF THE DRAWINGS

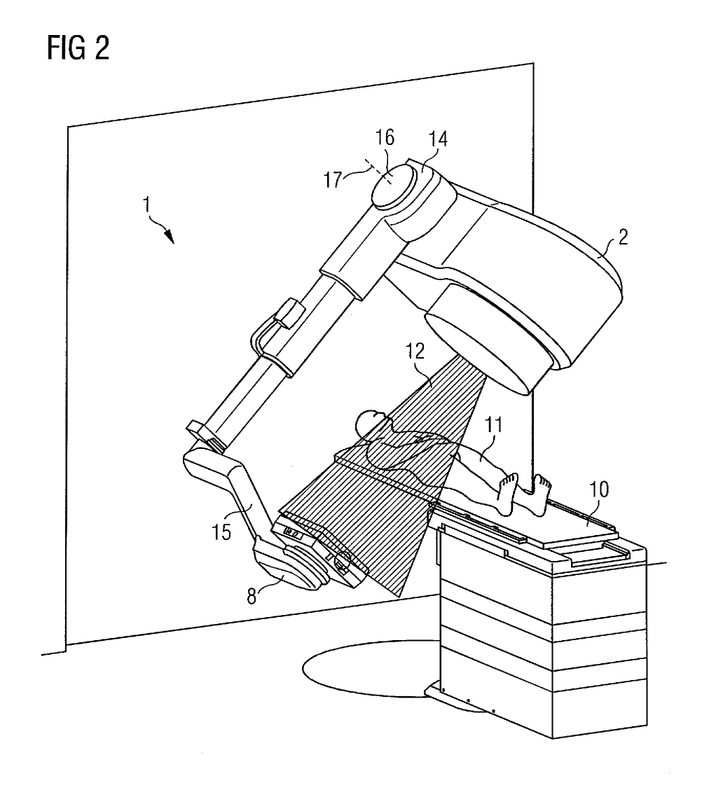

FIG. 2 shows one embodiment of a radiation therapy system including a first telescopic arm having an X-ray detector (e.g., an in-situ X-ray detector). The radiation therapy system 1 includes a radiation therapy module with a cantilever arm 2, in which a radiation source (not shown) for a therapeutic treatment beam (not shown) is at least partially located. During a treatment session, the therapeutic treatment beam may be directed onto an object 11 (e.g., a patient) lying on a patient bench 10. The cantilever arm 2 is secured on a kinematic control (not shown) such that the cantilever arm may be rotated about an isocenter, thus enabling the therapeutic treatment beam to be directed from different angles onto the isocenter. The radiation therapy system 1 also includes an X-ray imaging device, the X-ray source of which is embodied as a single-tank X-ray generator and generates X-radiation 12. The X-ray source is arranged in the cantilever arm 2 and is not shown in FIG. 2. A variable-length first telescopic arm 13 is arranged on the cantilever arm 2 and includes a first 14 end and a second end 15. The first telescopic arm 13 is arranged rotatably about a first axis of rotation 17 on a first securing point 16 on the cantilever arm 2. The first axis of rotation 17 stands perpendicular to the first securing point 16. An X-ray detector 8 of the X-ray imaging device is arranged on the second end 15 of the first telescopic arm 13 such that a diagnostic X-ray image of the object 11 may be produced. This arrangement of the X-ray source and the X-ray detector 8 makes inline-imaging possible. After taking one or more X-ray images, due to the variability of the length of the telescopic arm 13, the first telescopic arm 13 with the X-ray detector 8 is moved away from the imaging position and, for example, put into a park position so that the patient being examined is freely accessible to medical assistants.

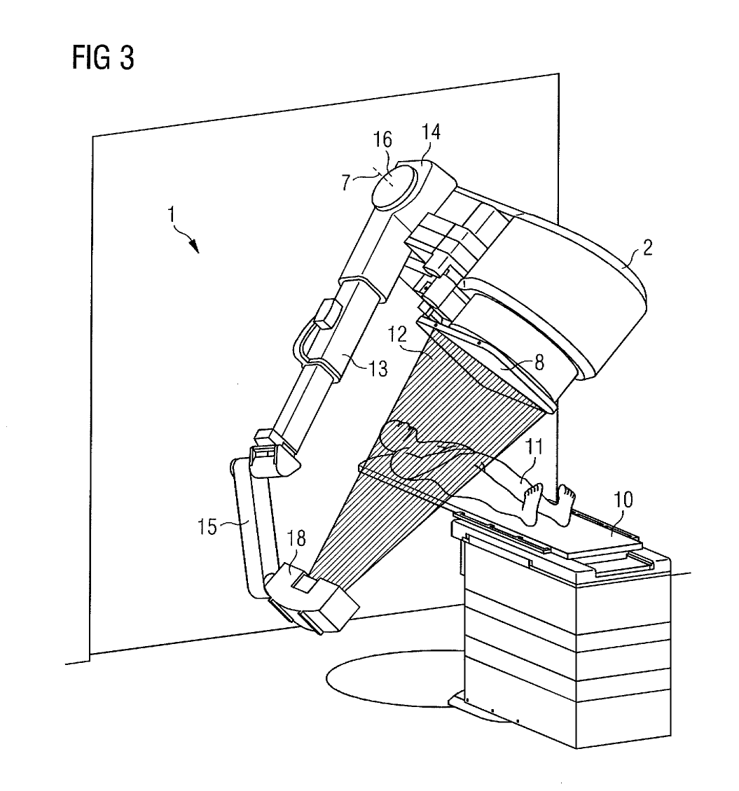

FIG. 3 shows one embodiment of a radiation therapy system with a first telescopic arm having an X-ray tube (e.g., an in-situ X-ray tube). The radiation therapy system 1 includes a radiation therapy module with a cantilever arm 2, in which the radiation source (not shown) for a therapeutic treatment beam (not shown) is at least partially located. During a treatment session, the therapeutic treatment beam is directed onto an object 11 (e.g., a patient) lying on a patient bench 10. The cantilever arm 2 is secured on a kinematic control (not shown) such that the cantilever arm 2 may be rotated about an isocenter, thus enabling the therapeutic treatment beam to be directed from different angles onto the isocenter. The radiation therapy system 1 also includes an X-ray imaging device. An X-ray detector 8 is arranged movably on the cantilever arm 2 and is swung into an outlet area of the therapeutic treatment beam. A variable-length first telescopic arm 13 is arranged on the cantilever arm 2 and includes a first 14 end and a second end 15. The first telescopic arm 13 is arranged rotatably about a first axis of rotation 17 on a first securing point 16 on the cantilever arm 2. The first axis of rotation 17 stands perpendicular to the first securing point 16. An X-ray source 18 in the X-ray imaging device, which generates X-radiation 12, is arranged on the second end 15 of the first telescopic arm 13 such that a diagnostic X-ray image of the object 11 may be produced when the X-ray detector 8 is swung into the outlet area of the treatment beam.

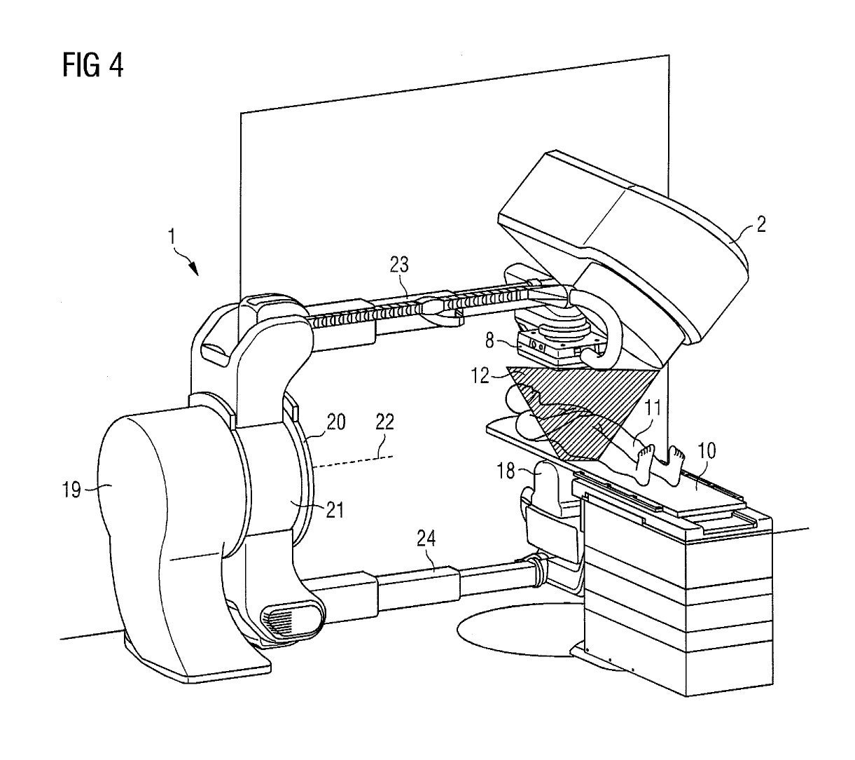

FIG. 4 shows one embodiment of a radiation therapy system with a first telescopic arm and a second telescopic arm. The radiation therapy system 1 includes a radiation therapy module with a cantilever arm 2, in which a radiation source (not shown) for a therapeutic treatment beam (not shown) is at least partially located. During a treatment session, the therapeutic treatment beam is directed onto an object 11 (e.g., a patient) lying on a patient bench 10. The cantilever arm 2 is secured on a kinematic control (not shown) such that the cantilever arm 2 may be rotated about an isocenter, thus enabling the therapeutic treatment beam to be directed from different angles onto the isocenter. A connecting element 20 on a second securing point 21 is arranged on a base carrier 19. The connecting element 20 is rotatable about a second axis of rotation 22 standing perpendicular to the second securing point 21. A variable-length first telescopic arm 23 and a variable-length second telescopic arm 24 are arranged on the connecting element 20. The radiation therapy system 1 also includes an X-ray imaging device with an X-ray detector 8 and an X-ray source 18 that generates X-radiation 12. The X-ray detector 8 is connected to an end of the first telescopic arm 23 facing away from the connecting element 20. The X-ray source 18 is connected to an end of the second telescopic arm 24 facing away from the connecting element 20. The X-ray detector 8 and the X-ray source 18 may be positioned such that a diagnostic X-ray image of the object 11 may be produced.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A radiation therapy system comprising:
a radiation therapy module comprising a cantilever arm, from which a therapeutic treatment beam is directable onto an object to be irradiated, the cantilever arm being secured on a kinematic control such that the cantilever arm is rotatable about an isocenter, thus enabling the therapeutic treatment beam to be directed from different angles onto the isocenter;
an X-ray imaging device comprising at least one X-ray source and an X-ray detector, wherein the at least one X-ray source is arranged in the cantilever arm so that X-radiation is directable in a direction of the therapeutic treatment beam; and
a first telescopic arm comprising a first end and a second end, the first end of the first telescopic arm being arranged rotatably on the cantilever arm, the X-ray detector being arranged on the second end, such that a diagnostic X-ray image of the object is producible.

2. The radiation therapy system as claimed in claim 1, wherein the first telescopic arm comprises at least two arm members connected to each other by articulated joints.

3. The radiation therapy system as claimed in claim 1, wherein a length of first telescopic arm is variable.

4. The radiation therapy system as claimed in claim 1, wherein the first telescopic arm is rotatable about a first axis that is perpendicular to a first securing point of the first telescopic arm on the cantilever arm.

5. A radiation therapy system comprising:
a radiation therapy module comprising a cantilever arm, from which a therapeutic treatment beam is directable onto an object to be irradiated and which is secured on a kinematic control such that the cantilever arm is rotatable about an isocenter, thus enabling the therapeutic treatment beam to be directed from different angles onto the isocenter;
an X-ray imaging device comprising an X-ray source and an X-ray detector, wherein the X-ray detector is arranged movably on the cantilever arm and is swingable into an outlet area of the therapeutic treatment beam; and
a first telescopic arm comprising a first end and a second end, the first end of the first telescopic arm being arranged rotatably on the cantilever arm, the X-ray source being arranged on the second end such that a diagnostic X-ray image of the object is producible when the X-ray detector is swung into the outlet area of the treatment beam.

6. The radiation therapy system as claimed in claim 5, wherein the first telescopic arm and the second telescopic arm each comprises at least two arm members connected to each other by articulated joints.

7. The radiation therapy system as claimed in claim 5, wherein lengths of first telescopic arm and the second telescopic arm are variable.

8. The radiation therapy system as claimed in claim 5, wherein the first telescopic arm is rotatable about a first axis that is perpendicular to a first securing point of the first telescopic arm on the cantilever arm.

9. A radiation therapy system comprising:
a radiation therapy module comprising a cantilever arm, from which a therapeutic treatment beam is directable onto an object to be irradiated and which is secured on a kinematic control such that the cantilever arm is rotatable about an isocenter, thus enabling the therapeutic treatment beam to be directed from different angles onto the isocenter;
an X-ray imaging device comprising an X-ray source and an X-ray detector; and
a base carrier comprising a rotatable connecting element, a first telescopic arm and a second telescopic arm being arranged on the rotating connecting element,
wherein the X-ray detector is arranged on an end of the first telescopic arm facing away from the rotatable connecting element, and the X-ray source is arranged on an end of the second telescopic arm facing away from the rotatable connecting element, and
wherein the X-ray detector and the X-ray source is positionable such that a diagnostic X-ray image of the object is producible.

10. The radiation therapy system as claimed in claim 9, wherein the first telescopic arm and the second telescopic arm each comprises at least two arm members connected to each other by articulated joints.

11. The radiation therapy system as claimed in claim 9, wherein lengths of first telescopic arm and the second telescopic arm are variable.

12. The radiation therapy system as claimed in claim 9, wherein the rotatable connecting element is rotatable about a second axis that is perpendicular to a securing point of the rotatable connecting element on the base carrier.

13. The radiation therapy system as claimed in claim 9, wherein the base carrier is arranged on the base and is connected to the base carrier.
